(19) United States
(12) Patent Application Publication
(10) Pub. No.: US 2004/0138619 A1
(43) Pub. Date: Jul. 15, 2004

(54) Retractable needle assembly for a catheter and which uses an elastomeric member to retract the needle

(76) Inventor: Bruce Leigh Kiehne, Queensland (AU)

Correspondence Address: HOFFMAN WASSON & GITLER, P.C, CRYSTAL CENTER 2, SUITE 522, 2461 SOUTH CLARK STREET, ARLINGTON, VA, 22202-3843, US

(21) Appl. No.: 10/475,854

(22) PCT Filed: Jun. 14, 2002
(86) PCT No.: PCT/AU02/00778

(30) Foreign Application Priority Data
Jun. 14, 2001 (AU) PR 5689

(51) Int. Cl. A61M 5/178

(52) U.S. Cl. 604/164.01; 604/167.03

(57) ABSTRACT

A retractable needle assembly can be used with a catheter and has a needle holder which is automatically shot back into the assembly body in a sharps safe manner when the canulla needle is moved forward into a vein. The needle is biased to withdraw by an elastic member that is stretch upon use of the needle assembly. Prior to use the elastic member is not stressed. Also disclosed is a valve assembly of a rigid hollow inner member and a compressible outer member. The outer has a self closing opening in register with an open end of the inner member to close the valve. The valve is opened by the outer member being moved away from said open end.

### FIELD OF THE INVENTION

This invention relates to a retractable needle assembly that can be used with a catheter, with the needle acting as the puncture needle, and where the needle can be retracted after use using an elastomeric member

### BACKGROUND ART

A catheter can comprise any of various tubular medical devices designed for insertion into veins or body cavities so as to permit injection or withdrawal of fluids or substances or to maintain the openness of a passageway. Catheters are widely used in hospitals for withdrawal of blood from a patient's body. In practice, the catheter comprises a puncture needle that is typically a conventional steel injection needle. About the puncture needle is a cannula. The cannula comprises a cannula needle through which the puncture needle passes. The cannula needle is typically formed from flexible plastic tube and does not have a sharp tip (as it does not itself puncture the body). The cannula needle is fitted to a plastic cannula base.

In use, the puncture needle penetrates into the person's body and passes into the person's vein. The cannula needle is then pushed forwardly to also enter into the person's vein. The puncture needle can then be retracted. A tube or other type of container can then be attached to the cannula base to collect the blood.

As the puncture needle is retracted entirely out of the cannula needle, it presents a sharps hazard, and also a biohazard due to contamination of the needle with body fluids. For this reason, various attempts have been made to reduce the sharps hazard and also the biohazard.

Various known arrangements use tip protection to reduce the hazard. The tip protection comprises some form of cover that is placed over the sharp tip of the puncture needle after the needle has been used. Attachment of these types of tip protectors can in itself create a hazard.

It is also known to provide a shoot back needle for a catheter where the puncture needle is biased by a helical spring to shoot back into some form of holding body after the cannula needle has been inserted. The advantage of this arrangement is that the needle can be removed without needing to be touched by a person.

It is also known to use a vacuum to shoot back (or perhaps more correctly, to suck back) the puncture needle after use. In one known arrangement, the needle is attached to some form of base member that is under permanent vacuum. That is, the puncture needle assembly is purchased with the assembly already under vacuum. This raises a host of potential difficulties. For instance, it is envisaged that air may slowly leak into the holding body over time to gradually decrease the vacuum. This could typically occur if the puncture needle assembly is stored for a period of time before use. If vacuum is lost, this is not at all evident by a user until it becomes obvious that the needle will not shoot back properly and this can then create a sharps hazard or a biohazard. Another disadvantage is that transportation, handling and storage of such permanently depressurised assemblies may create safety hazards. For instance, an assembly could conceivably implode if handled roughly or accidentally knocked, dropped and the like.

To overcome the disadvantage is associated with the existence of a permanent vacuum, it is also known to provide a shoot back needle arrangement, where the needle is shot back into a body using a vacuum, but where the vacuum is created only at the point of use of the device. That is, during storage and transportation, the device is not under vacuum. Attaching the puncture needle to some form of plunger body typically creates a vacuum, and sliding the plunger body through an outer cylindrical body to create a vacuum as the plunger body is moved. The plunger body is typically releasably held to the outer cylindrical body by some form of catch or latch member. Release of the plunger body will cause it to shoot back into the outer cylindrical body drawing back the puncture needle in the process. This arrangement also has some disadvantages, including the requirement to accurately control the pressure reduction to ensure that the puncture needle does not shoot back to rapidly, to ensure that there is no pressure loss once the device is cocked and prior to use of the device. Another disadvantage with these known devices is that the needle would tend to shoot back as soon as the catheter was pushed forwardly. It is necessary to ensure that the catheter can be pushed forwardly in a smooth and even manner to prevent the catheter from becoming misaligned in the persons vein, body cavity and like. The almost immediate shoot back mechanism of the puncture needle tended to upset the smooth and even forward movement that could result in the catheter becoming misaligned.

Another disadvantage with catheters is in preventing blood or bio fluid from passing through the catheter before a container or other desired device could be attached to the catheter to collect the blood or bio fluid. There would be an advantage in providing some form of valve means to prevent undesirable flow of a bio fluid through the catheter.

### OBJECT OF THE INVENTION

The present invention is directed to a retractable needle assembly which can be used with a catheter, and where the needle can be retracted in a “sharps safe” manner, and where the needle is retracted using an elastomeric member, thereby doing away with the need to have a vacuum to draw back the needle, or some form of helical spring to draw back the needle.

It is an object of the invention to provide a retractable needle assembly that may at least partially overcome the abovementioned disadvantages or provide public with a useful or commercial choice.

In one form, the invention resides in a retractable needle assembly comprising:

an outer elongate hollow body member which has a rear end and a front end, the front end being open,

a needle assembly which comprises a needle which is attached to a needle body, the needle body adapted for sliding movement along the hollow body member from adjacent the rear end to adjacent the front end, with the needle projecting from the front end when the needle body is adjacent the front end, a collar, the needle assembly being releasably attachable to the collar, the collar adapted for sliding movement along the hollow body member from adjacent the rear end to adjacent the front end,

needle assembly retraction means comprising elastomeric means, the elastomeric means being in a substantially unstretched state when the needle assembly is adjacent the rear end of the outer body, and in a stretched state when the needle assembly is adjacent the front end of the outer body,

holding means to hold the collar against the bias of the elastomeric means, when the collar is adjacent the front end of the outer body member, and,

release means to release the needle assembly from the collar to allow the needle assembly to be shot back into the outer body by retraction of the elastomeric means from its stretched state to its substantially unstretched state.

In a broader form, the invention resides in a retractable needle assembly which comprises an outer elongate body, a needle assembly containing a needle and a needle body, the needle assembly being moveable along the body from a rearward portion of the body to a forward portion of the body, and retraction means to retract the needle assembly from its forward portion to its rearward portion, the retraction means comprising elastomeric means.

In another form, the invention resides in a valve assembly insertable into a cannula base, the cannula base having a forward end supporting the cannula needle, a substantially hollow main base portion, and an open rear end into which a luer tip of a syringe can pass, the valve assembly having an inner part and an outer part, the inner part comprising a substantially rigid tubular member one end of which is in fluid communication with the cannula needle, and the other end of which is free, the outer part comprising a resilient compressible tubular member adapted to extend over the inner part and having a sealing end member, the end member being moveable between a sealing position where the end member extends over and seals the other end of the inner part, and an open position where the end member has been pushed away from the other end of the inner part, thereby allowing fluid to flow through the inner part, the end member being provided with a self-closing opening, the opening being biased into a naturally closed position.

In a broader form, the invention relies in a valve assembly which comprises an inner part and an outer part, the inner part comprising a rigid hollow member having an open end, the outer part comprising a compressible member having a sealing end which is formed with a self-closing opening, the outer part being moveable between a sealing position where the sealing end extends over the open end of the hollow member and the self-closing opening is closed, and an open position, where the sealing end has moved away from the open end of the hollow member.

While the retractable needle assembly will be described with reference to its use with a catheter, it is envisaged that the invention need not be limited to only this use.

The outer body member may comprise a hollow elongate tube. The length of the tube may vary but will typically be between 30-200 mm. The outer body member is typically cylindrical and may have a diameter of between 10-30 mm. The outer body member is typically formed of plastics material. The outer body member has a rear end and a front end, the front end being open. Suitably, the rear end is also open. The rear end may be formed with a peripheral upwardly extending lip.

The outer body member may be formed with an elongate slot or recess. The elongate slot or recess may extend through the wall of the outer body member and substantially along the outer body member, and may be substantially straight. The elongate slot or recess typically extends from the rear end of the outer body member to, but spaced inwardly from, the front end of the outer body member. Suitably, a pair of such elongate slots or recesses is provided. These may extend in a diametrically opposite manner relative to each other. A forward part of the outer body member may also be provided with the holding means to hold the collar in its forward position. The holding means may be in the form of a catching finger that may be formed integrally with the main body member, the catching finger able to catch or lock the collar in the forward position.

A needle assembly is provided which has a needle (typically a conventional steel needle) attached to a needle body. The needle assembly is able to move along the outer body member from adjacent the rear end of the outer body member to adjacent the front end of the outer body member. Suitably, the needle body is sized to fit within the outer body member and to enable the needle body to slide through the outer body member. It is not necessary for there to be any vacuum sealing between the needle body and the outer body member.

The needle body functions to support the needle. Suitably, the needle body is substantially hollow and is provided with a viewable flash chamber to allow a flash of blood (or other type of bio fluid) to be seen which signifies successful penetration of the needle into the person's vein. An air permeable (but blood impermeable) partition is preferably provided in the hollow body and spaced from one end of the body that may act as a vent. The needle body may be provided with an attachment means, for instance in the form of a hook, which facilitates attachment of the needle body to the needle assembly retraction means.

The assembly may comprise a collar. The collar, if provided, is adapted for sliding movement along the outer body member from adjacent the rear end of the body member to adjacent the front end of the body member. The collar may be formed of plastics material. The collar may be substantially tube like in configuration, or may comprise a substantially U shaped collar. The collar is designed such that the needle body can be supported, or attached to the collar. Suitably, the needle body is able to extend substantially within the collar. The collar and/or the needle assembly may be provided with releasable attachment means such that movement of the collar towards the front end of the outer body member causes the needle assembly to also be moved towards the front end of the outer body member.

The collar may be provided with finger grippable portions to allow an operator to push the collar forwardly along the outer body member. The finger grippable portions may extend through the slot or recess in the outer body member to enable an operator to grip and push the collar forwardly along the outer body member. The collar may be provided with the release means that can release the needle assembly from engagement with the collar.

A needle assembly retraction means is provided. This comprises an elastomeric means. In one form, the elastomeric means may comprise an elastomeric member. One or more of such elastomeric members may be provided. In the elastomeric members may comprise elongate linear members, elongate non-linear members and the like.

Suitably, the elastomeric means forms part of an end cap. The end cap may cap an otherwise open rear end of the outer body member. The end cap and the at least one elastomeric member may be formed integrally and may comprise any suitable elastomeric member or compound. Suitable elastomeric means may comprise natural or artificial rubbers, neoprenes, silicones and the like. The end cap may be shaped such that it provides a slight compressive force to the rear end of the outer body member when attached thereto.

If the outer body member is provided with one or more longitudinal slots or recesses, the end cap can function to slightly compresses or narrow the diameter of the outer body member to function as a brake means to control the speed of retraction of the needle assembly.

The elastomeric member is operatively connected to the needle holder, and is typically connected to the needle body, for instance by attachment about a hook on the needle body. Suitably, one end of the elastomeric member is operatively connected to the needle holder, while the other end of the elastomeric member is secured to or formed integrally with the end cap.

The elastomeric means can be stretched or extended from a substantially unstretched state when the needle assembly is adjacent the rear end of the outer body member, and an at least partially stretched state when the needle assembly is adjacent the front end of the outer body member.

The assembly may be used in association with a cannula. The cannula typically comprises a cannula needle and a cannula body. The cannula may be of conventional design, and/or may contain a valve assembly as described above. The cannula may have a rear end formed with any type of necessary attachment means to allow the cannula to be attached to a collection chamber or some other required device. Thus, the rear end may be threaded, formed with a luer taper, or have any other type of convenient or required means for attachment to a required device.

### BRIEF DESCRIPTION OF THE DRAWINGS

An embodiment of the invention will be described with reference to the following drawings in which:

FIG. 1. Illustrates an exploded view of the various components of the assembly.

FIG. 2. Illustrates a section view of the assembly in the rest position.

FIG. 3. Illustrates a side view of the assembly in the rest position.

FIG. 4. Illustrates a side section view of the apparatus in the rest position.

FIG. 5. Illustrates a plan view of the assembly in the cocked position.

FIG. 6. Illustrates a side view of the assembly in the cocked position.

FIG. 7. Illustrates a side section view of the assembly in the cocked position.

FIG. 8. Illustrates a side view of the assembly where the needle has been shot back into the outer body member.

FIG. 9. Illustrates a section view of FIG. 8.

FIG. 10. Illustrates a cannula having a valve assembly in the sealing position.

FIG. 11. Illustrates the cannula of FIG. 10 with the valve assembly in the open position.

### BEST MODE

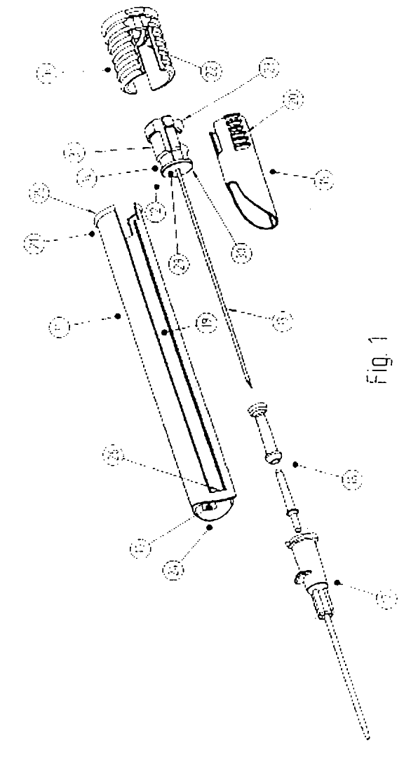

Referring initially to FIG. 1 there is illustrated the various parts of the retractable needle assembly. The assembly comprises an outer body member 11, a needle assembly 12 which comprises a steel puncture needle 13 and a needle body 14, a separately formed collar 15, a needle assembly retraction means part of which comprises an end cap 16, and a cannula 17 which is more or less of standard design, but which contains an internal valve assembly 18.

In summary, and according to an embodiment of the invention, the needle body 14 is held within collar 15. Collar 15 (and the contained needle body 14) fits within outer body member 11. Outer body member 11 is formed with opposed elongate slots 19, and collar 15 contains opposed extending finger grippable profiles 20 which extend through slots 19. End cap 16 is fitted to the rear end 21 of outer body member 11. End cap 16 contains an elastomeric member 22 in the form of a U shaped elastic band or cord which fits about a hook 23 on needle body 14. A cannula 17 is fitted over needle 13 in the usual manner. In use, an operator grips profiles 20 and pulls collar forwardly along outer body member from the rear end 21 to the front end 24. In doing so, needle body 14 is pulled forwardly and causes elastomeric member 22 to stretch. Collar 15 when pushed to the forward position catches on the outer body member 11 (described in greater detail below) and is held in position. The needle assembly is now in the cocked position. To retract the needle assembly, cannula 17 is pushed forwardly into the person's vein or body cavity, and this motion releases the needle assembly from attachment to collar 15 (described in greater detail below) which in turn causes the needle assembly to be pulled back into the body member by contraction of the elastomeric member 22 which stays attached to hook 23. Collar 15 does not retract with the needle assembly but stays attached to a forward part of the outer body member 11.

The various parts of the needle assembly will now be described in greater detail.

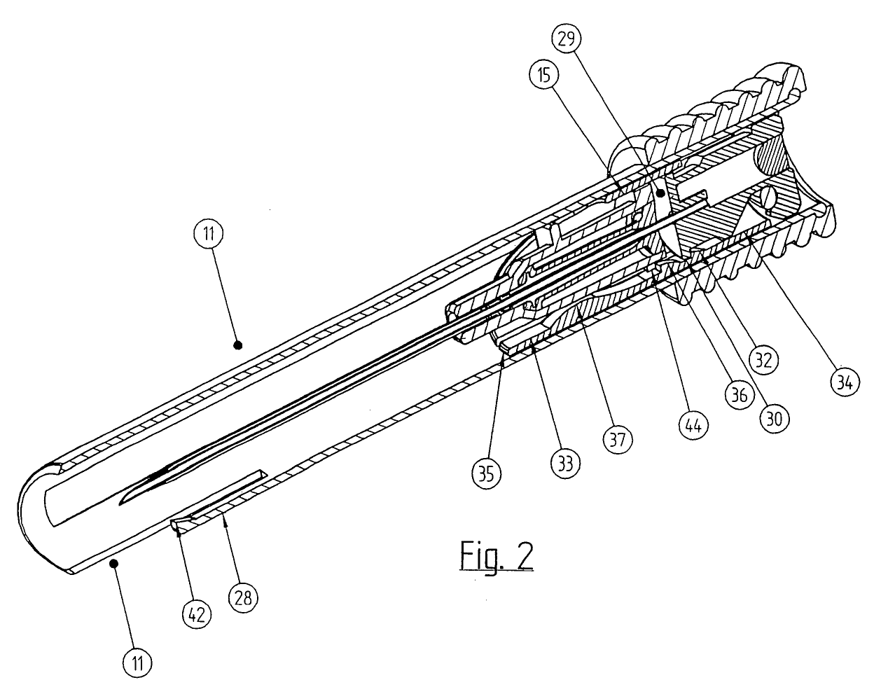

Outer body member 11 comprises an elongate hollow plastic tube which can have any suitable length and diameter, but would include lengths of between 5-20 cm and diameter is of between 5-30 mm. Outer body member 11 has a rear end 21 and a front end 24. Both ends are open. Rear end 21 is formed with a thickened lip 25 to facilitate attachment of end cap 16. Outer body member is formed with a pair of diametrically opposite longitudinal straight slots 19. Slots 19 are open at the rear end 21 of outer body member 11, but terminate at a position spaced inwardly from front end 24 to provide a stop 26 the reason for which will be described in greater detail below, but which prevents collar 15 from being pulled entirely out of the outer body member 11. By having the slots open at the rear end 21, it is possible to squeeze or compress this end of the outer body member to slightly reduce the internal diameter of the outer body member. The compression occurs when end cap 16 is fitted to the rear end 21 of the outer body member 11, as the end cap is designed to cause the outer body member to become slightly compress. The reason for this will be described in greater detail below, but is to slow down the speed that the needle assembly is retracted back into the outer body member. This is achieved by increasing the frictional contact between the needle assembly and the outer body member by reducing the diameter of the outer body member. The forward part of the outer body member 11 contains a cutout 27 which is best illustrated in FIG. 2, and a catching finger 28 (see FIG. 2) is positioned in this cutout 27, the catching finger 28 being formed integrally with outer body member 11, and functioning to engage with collar 15 to prevent collar 15 from being retracted. This will be described in greater detail below.

Needle assembly 12 comprises a puncture needle 13 which may be a conventional steel needle, and a needle body 14 of special design. Needle body 14 has a front circular dislike portion 29 which has a peripheral edge 30 which functions to releasably lock the needle body into engagement with collar 15 in a manner which will be described in greater detail below. Needle body 14 has a viewable flash chamber 31 formed of clear material. Flash chamber 31 comprises a hollow internal portion of needle body 14 and is in communication with needle 13. The rear end of flash chamber 31 is plugged with a blood impermeable but gas permeable plug to provide a pressure equalising effect. Needle body 14 contains an attachment means in the form of hook 23 which attaches to the elastomeric member 22 on end cap 16.

A collar 15 is provided in the embodiment. Collar 15 comprises a separate curved or tubular member formed of plastic material. The outer configuration of collar 15 is such that collar 15 can slide inside outer body member 11. Collar 15 is provided with a pair of diametrically opposite extending profiles 20 which are formed integrally with the collar and which provide finger grippable portions to an operator to allow the operator to push the collar along outer body member 11. Profiles 20 are positioned such that they extend through slots 19 in the outer body member 11. Collar 15 extends about and supports the needle assembly and cannula 17 in a manner best illustrated in FIG. 2. In particular, collar 15 functions to releasably attach the needle body 14 to the collar 15. This releasable attachment means is best illustrated in FIG. 2, and comprises a finger member 33 which sits inside a cutout in the collar. Finger member 33 is attached to a rear part of collar, and generally at the area referred to by reference numeral 34 in FIG. 2. The front end 35 of finger member 33 is free. Finger member 33 can therefore be seen as being cantilevered from attachment area 34. Finger member 33 contains a first tooth 32, a second tooth 36, and a gradual raised portion 37. The function of these parts will be described below. However, first tooth 32 engages with the edge 30 of portion 29 and this is functions to releasably lock the needle body to the inside of collar 15.

The needle assembly according to the embodiment has a holding means to hold the collar against the bias of the elastomeric member. The holding means in the embodiment comprises the catching finger 28.

The needle assembly according to the embodiment has a release means to release the needle assembly from the collar. In the embodiment, the release means comprise parts of finger member 33 and will be described in greater detail below.

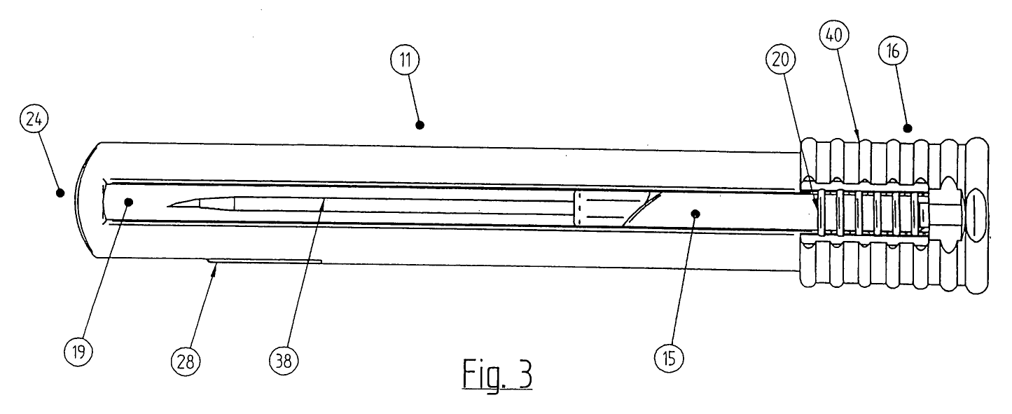

In use, the needle assembly is in the rest position illustrated in FIGS. 2-4. In this rest position, a cannula 17 containing its cannula needle 38 is slid over puncture needle 13 with the rear end of the cannula 39 abutting portion 29 which forms part of needle body 14 and locating about second tooth 36. Tooth 36 serves as a key to locate the catheter body in an upright position in packaging and first use position, such that finger grip ridge 43 is presented at the top. Catheter body lip 44 typically carries a slot, through which tooth 36 engages. Tooth 36 is shaped in such a way that the engagement of the tooth into the slot, providing the keying function, does not interfere with the triggering mechanism. This is required in use to enable the user to twist the catheter on the puncture needle once the catheter body has been pushed clear of tooth 36 and the production “seal” between the steel puncture needle and the plastic sheath is broken. This twisting is a habitual pattern of use amongst many users.

Needle body 14 is positioned in the rear end of outer body member 11 and within the end cap 16. The puncture needle 13 sits within the confines of outer body member 11 and does therefore not present a sharps hazard. End cap 16 is formed with grip enhancing outer profiles 40. The end cap is formed with opposed slots to allow the grip enhancing profiles 20 on collar 15 to be grippable by an operator's fingers when the collar is in this retracted position.

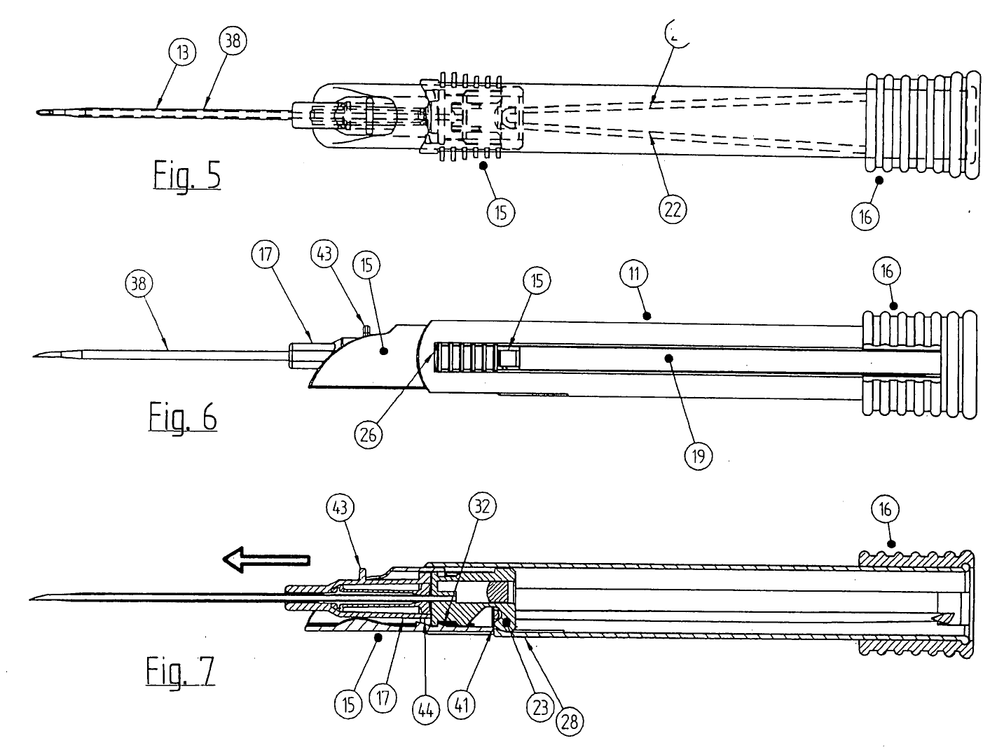

To operate the needle assembly, collar 15 is moved forwardly from the position illustrated in FIGS. 2-4, to the position illustrated in FIGS. 5-7. As collar 15 is moved forwardly, its attached body 14 is also moved forwardly, as needle body 14 is releasably locked to collar 15 via edge 30 engaging against tooth 32. The forward movement of collar 15 begins to stretch the elastomeric member 22, this being best illustrated in FIG. 5. Collar 15 moves partially through the open front end 24 of outer body member 11 such that a forward part of collar 15 projects from the open front end, this being best illustrated in FIG. 6. Collar 15 is prevented from being pulled entirely out of the front end 24 of outer body member 11 by stop 26 preventing profiles 20 (attached to collar 15) from moving further forward. When collar 15 is in this forward position, catching finger 28 (see FIG. 7) catches against a rear wall 41 of collar 15 to lock collar 15 into its forwardly position with a positive and audible click to indicate successful operation.

Catching finger 28 has a ramped portion 42 (best illustrated in FIG. 2), and this will cause catching finger 28 to momentarily bend downwardly as the collar rides over ramped portion 42, and then catching finger 28 will snap back into its original position to lock collar 15 into the forward position illustrated in FIGS. 5-7. This position can be seen as the cocked position where the needle holder is being biased back by elastomeric member 22, but is prevented from doing so by being locked to collar 15, with collar 15 being locked into the front area of outer body member 11.

At this cocked position, the needle assembly can now be placed against a person skin, and the front of steel needle 13 (which projects from the cannula needle) can be pushed into a person's vein or body cavity. Thereafter, the operator begins to push cannula 17 forwardly such that the flexible cannula needle 38 rides over the steel needle 13 and into the person's vein or body cavity. This technique is entirely conventional. The cannula typically has a projection 43 against which the operator's finger can push to push the cannula forwardly in a smooth and gentle manner.

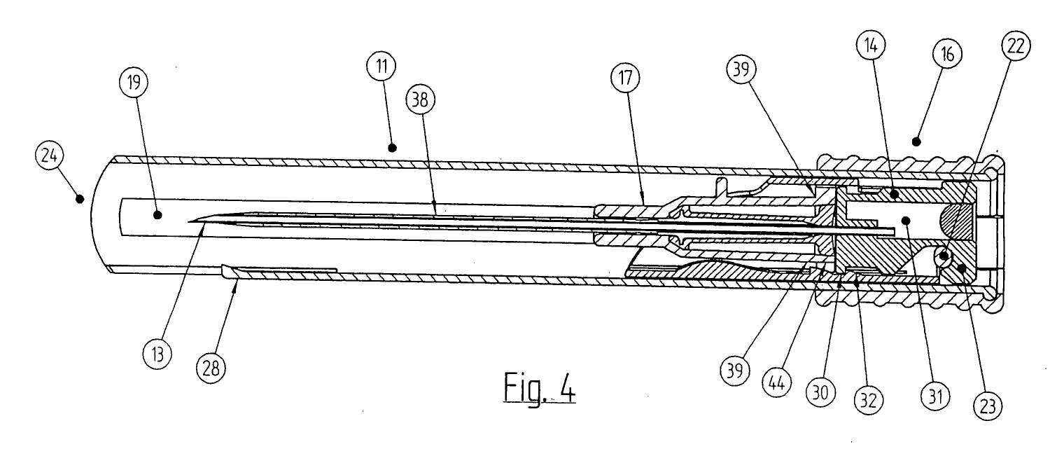

At this point, another feature of the present invention becomes apparent. Conventionally, shoot back needles tend to operate as soon as the cannula is pushed forwardly, and this can result in a non-smooth forward pushing of the cannula. In the present embodiment, the cannula is able to be pushed forwardly by a distance sufficient to allow it to be properly inserted into the persons body before the needle assembly shoot back. This distance can vary but is typically between 7-10 mm. In the embodiment, this is achieved by the configuration of finger member 33. Referring to FIG. 7, cannula 17 is pushed forwardly in the direction of the arrow. Cannula 17 has an end wall formed with a small lip 44 the lip being best illustrated in FIG. 2, and FIG. 4. As the cannula begins to move forwardly, small lip 44 begins to ride along finger member 33. Initially, finger member 33 has a flat portion between lip 44 and raised portion 37. This flat portion allows the cannula to travel forwardly without triggering the shoot back mechanism. Further forward movement of the cannula will cause lip 44 to begin to ride up and along raised portion 37. This in turn begins to cause finger member 33 to be pushed downwardly. The downward movement begins to release the edge 30 of needle body 14 against engagement with the first tooth 32. At some stage, finger member 33 is pushed downwardly sufficiently to release the needle body from engagement with the first tooth 32, after which the needle body containing the attached steel needle 13 is pulled back into outer body member 11 by contraction of the stretched elastomeric member 22. This action therefore comprises the shoot back mechanism and is triggered by forward movement of the cannula by the operator. This provides advantages over conventional devices where a separate trigger is provided which must be depressed by the operator. In the embodiment, the trigger forms part of the forward movement of the cannula. The shoot back operation does not triggered immediately, but only trigger is when the cannula has been moved forward sufficiently such that lip 44 on the cannula rides over raised portion 37.

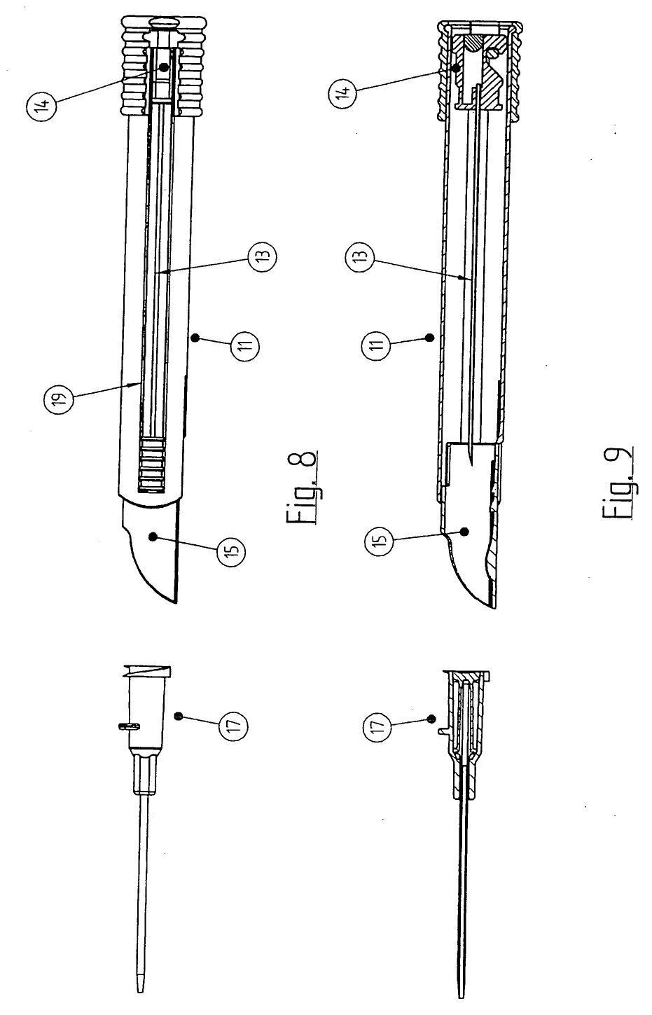

Referring to FIGS. 8 and 9, the needle holder 14 and needle 13 have been shot back into the confines of outer body member 11 such that the used needle 13 does not project from the outer body member and the remaining collar 15 and therefore prevents a sharps hazard. Collar 15 in this position forms a reduced diameter protective ring around the needle tip, ensuring that the needle cannot be extended sideways through the side slots.

The end cap 16 slightly compresses the rear of outer body member 11 to cause a slight reduction in the internal diameter. This functions as a braking mechanism by increasing the fictional contact between the needle holder and the internal wall of outer body member 11 as the needle holder is shot back.

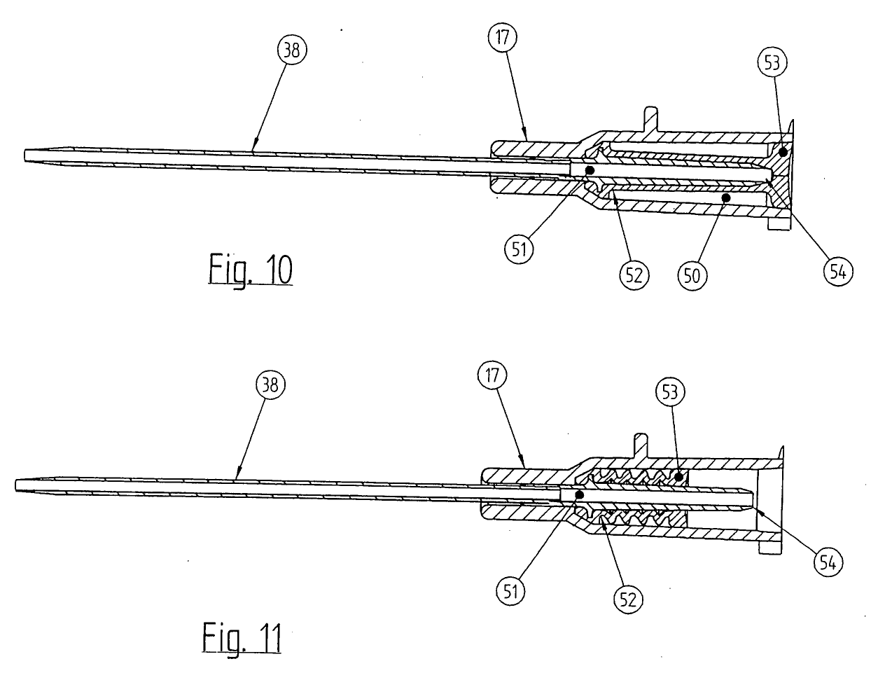

The cannula may, in an embodiment, be formed with a valve assembly. An embodiment of the valve assembly is illustrated in FIGS. 10-11. Valve assembly 50 comprises two parts being an inner part and an outer part. The inner part comprises a steel hollow tube 51. Tube 51 has a diameter which is larger than the outer diameter of the steel puncture needle which means that the steel puncture needle can pass through hollow tube 51. The outer part comprises an elastomeric compressible sleeve 52 which sits about tube 51, and which is moveable between an extended position illustrated in FIG. 10, and a compressed position illustrated in FIG. 11. Sleeve 52 has a sealing end face 53. End face 53 is formed with a slit or other type of opening. End face 53 when in the extended position illustrated in FIG. 10 seals the otherwise open end 54 of hollow tube 51. In this position, blood or other bio fluid is unable to pass through and leak out of the end of the cannula. When end face 53 is in the compressed position illustrated in FIG. 11, it rides up over tube 51 and therefore the open end 54 of tube 51 is unrestricted. The slit or other type of opening in sealing end face 53 is designed to be naturally biased into the closed position. The sealing end face 53 can be given a slightly concave configuration to facilitate closure of the slit. Sealing end face 53 is pushed into the compressed state illustrated in FIG. 11 upon insertion of a lure tip of a syringe, or other type of device into the end of cannula 17.

It should be appreciated that various other changes and modifications may be made to the embodiment described without departing from the spirit and scope of the invention.

1. A retractable needle assembly comprising:
an outer elongate hollow body member which has a rear end and a front end, the front end being open,
a needle assembly which comprises a needle which is attached to a needle body, the needle body adapted for sliding movement along the hollow body member from adjacent the rear end to adjacent the front end, with the needle projecting from the front end when the needle body is adjacent the front end,
a collar, the needle assembly being releasably attachable to the collar, the collar adapted for sliding movement along the hollow body member from adjacent the rear end to adjacent the front end,
needle assembly retraction means comprising elastomeric means, the elastomeric means being in a substantially unstretched state when the needle assembly is adjacent the rear end of the outer body, and in a stretched state when the needle assembly is adjacent the front end of the outer body,
holding means to hold the collar against the bias of the elastomeric means, when the collar is adjacent the front end of the outer body member, and,
release means to release the needle assembly from the collar to allow the needle assembly to be shot back into the outer body by retraction of the elastomeric means from its stretched state to its substantially unstretched state.

2. The assembly of claim 1, wherein the outer body member comprises a hollow elongate tube formed with an elongate slot which extends through the wall of the outer body member and substantially along the outer body member.

3. The assembly of claim 2, wherein a forward part of the outer body member is provided with the holding means to hold the collar in its forward position.

4. The assembly of claim 3, wherein the holding means comprises a catching finger formed integrally with the main body member, the catching finger able to catch or lock the collar in the forward position.

5. The assembly of claim 1, wherein the needle body is sized to fit within the outer body member and to enable the needle body to slide through the outer body member.

6. The assembly of claim 5, wherein the needle body is provided with an attachment means to attach the needle body to the needle assembly retraction means.

7. The assembly of claim 1, wherein the collar and the needle assembly are releasably connected together when the collar moves from adjacent the rear end of the hollow body member to adjacent the front end of the body member.

8. The assembly of claim 7, wherein the collar is provided with finger grippable portions to allow an operator to push the collar forwardly along the outer body member, the finger grippable portions extending through a slot or recess in the outer body member to enable an operator to grip and push the collar forwardly along the outer body member.

9. The assembly of claim 8, wherein the collar is provided with the release means that can release the needle assembly from engagement with the collar.

10. The assembly of claim 1, wherein the elastomeric means comprises an elastimeric member which forms part of an end cap, the end cap being attached to the rear end of the outer body member.

11. The assembly of claim 10, wherein the elastomeric member is attached to the needle body.

12. The assembly of claim 9, wherein the release means comprises a finger member attached to the collar and which can be depressed from a first position where the finger locks the collar to the needle assembly, and a second position where the needle assembly is released from the collar.

13. The assembly of claim 12, wherein a canulla is attached to the needle assembly.

14. The assembly of claim 13, where forward movement of the canulla relative to the needle assembly causes depression of the finger member to release the needle assembly from the collar.

15. The assembly of claim 13 wherein the canulla has a base, a valve assembly in the cannula base, the cannula base having a forward end supporting the cannula needle, a substantially hollow main base portion, and an open rear end into which a luer tip of a syringe can pass, the valve assembly having an inner part and an outer part, the inner part comprising a substantially rigid tubular member one end of which is in fluid communication with the cannula needle, and the other end of which is free, the outer part comprising a resilient compressible tubular member adapted to extend over the inner part and having a sealing end member, the end member being moveable between a sealing position where the end member extends over and seals the other end of the inner part, and an open position where the end member has been pushed away from the other end of the inner part, thereby allowing fluid to flow through the inner part, the end member being provided with a self-closing opening, the opening being biased into a naturally closed position.

16. A retractable needle assembly which comprises an outer elongate body, a needle assembly containing a needle and a needle body, the needle assembly being moveable along the body from a rearward portion of the body to a forward portion of the body, and retraction means to retract the needle assembly from its forward portion to its rearward portion, the retraction means comprising elastomeric means.

17. A valve assembly which comprises an inner part and an outer part, the inner part comprising a rigid hollow member having an open end, the outer part comprising a compressible member having a sealing end which is formed with a self-closing opening, the outer part being moveable between a sealing position where the sealing end extends over the open end of the hollow member and the self-closing opening is closed, and an open position, where the sealing end has moved away from the open end of the hollow member.
